(19) United States
(12) Patent Application Publication
(10) Pub. No.: US 2006/0252683 A1
(43) Pub. Date: Nov. 9, 2006

(54) Preparation of a peptide compound (SYRAPRO-2000) and its use for the activation of protein phosphatase-2A1 enzyme

(76) Inventor: Hin Young Lim Tung, Long Island City, NY (US)

Correspondence Address: DR. H.Y. Lim Tung, P.O.Box 6009, Astoria, NY, 11106, US

(21) Appl. No.: 11/121,132

(22) Filed: May 4, 2005

(51) Int. Cl. A61K 38/17

(52) U.S. Cl. 514/12

(57) ABSTRACT

This invention relates to the preparation of a peptide compound (SYRAPRO-2000), its use to activate protein phosphatase-2A enzymes for the dephosphorylation of proteins in vitro and in the cell and its use for inhibiting cell proliferation and induction of death of brain cancer cells and other cancer cells and therefore the treatment of proliferative disorder such as brain cancer and other forms of cancer. Drugs that target the enzymes of the protein phosphorylation/dephosphorylation apparatus of the cell are mostly inhibitor compounds. The present invention describes for the first time the use of an activating compound of a protein phosphatase to modulate the activity of a cell. In this invention, the peptide compound activator of protein phosphatase-2A1 (SYRAPRO-2000) is used to activate protein phosphatase-2A1, thereby causing inhibition of cell proliferation and induction of cell death of transformed T cells and brain cancer cells.

### STATEMENT REGARDING FEDERALLY SPONSORED RESEARCH OR DEVELOPMENT

This invention was not made by an agency of the United States Government or under a contract with an agency of the United States Government

### OTHER REFERENCE

Tung, H. Y. L., Resink, T. J., Hemmings, B. A., Shenolikar, S. and Cohen, P. (1984) The catalytic subunits of protein phosphatase-1 and protein phosphatase-2A are distinct gene products. Eur. J. Biochem. 138, 635-641.

Tung, H. Y. L., Alemany, S. and Cohen, P. (1985) The protein phosphatases involved in cellular regulation: Purification, subunit structure and properties of protein phosphatase-2A₀,-2A₁ and -2A₂ from rabbit skeletal muscle. Eur. J. Biochem. 148, 253-363.

Pelech, S. and Cohen, P. (1985). “The protein phosphatases involved in cellular regulation. 1. Modulation of protein phosphatases-1 and 2A by histone H1, protamine, polylysine and heparin.” Eur. J. Biochem. 148: 245-251.

Tung, H. Y. L., De Rocquigny, H., Cayla, X., Zhao, L.-J., Roques, B. and Ozon, R. (1997) Direct activation of protein phosphatase-2A₀ by HIV-1 encoded protein complex NCp7:vpr.) FEBS Letts. 401,97-201.

### BACKGROUND OF THE INVENTION

Protein phosphatase-2A enzymes are key enzymes of the protein phosphorylation/dephosphorylation of the cell (Ingebritsen 1983; Cohen 1989; Jannssens 2001). One form of protein phosphatase-2A termed protein phosphatase-2A1, is involved in the dephosphorylation of proteins that are important for the control of cell maintenance, cell proliferation and cell death (Tung 1984; Tung 1985; Meijer 1986; Deng 1998; Chiang 2002; Chiang 2003). It has a basal activity in the cell that is necessary for cell maintenance (Ingebritsen 1983; Ingebritsen 1983). Low activity of protein phosphatase-2A1 is associated with proliferation while high activity is associated with cell death (Sontag 1993; Sontag 1997; Millward 1999; Petritsch 2000; Li 2001; Garcia 2003; Morrow 2004; Janoo 2005). Activating molecules of protein phosphatase-2A1 will be useful in inhibiting cell proliferation and induction of death of cancer cells.

There is currently no known physiological activators of protein phosphatase-2A. Previous work has shown that artificial activators of protein phosphatase-2A are basic in character (e.g protamine and histone (Pelech 1985; Tung 1985). However, they are not permeable to the cell membrane. From the literature, HIV-1 Vpr may be an activator of protein phosphatase-2A1 (Emerman 1996; Tung 1997). However, full length HIV-1 Vpr was tested and it was shown not to activate protein phosphatase-2A1 (Tung 1997; Janoo 2005). Analysis of the C-terminal fragment of HIV-1 Vpr indicates that the C-terminal fragment of HIV-1 Vpr may be an activator of protein phosphatase-2A1.

The present invention is concerned with the preparation of a peptide compound (SYRAPRO-2000), its use to activate protein phosphatase-2A1 in vitro and in intact cells, and its use in inhibiting cell proliferation and inducing death of cancer cells. Currently, most drugs that target the enzymes of the protein phosphorylation/dephosphorylation apparatus of the cell are inhibitor compounds (Cohen 2002; McCluskey 2002; Melnikova 2004). The present invention which is based on a published paper (Janon 2005) is the first to describe the use of an activating compound of a protein phosphatase to modulate the activity of a cell which in this invention is: inhibition of cell proliferation and induction cell death of transformed T cells and brain cancer cells.

### BRIEF SUMMARY

The rational use of a peptide compound activator of protein phosphatase-2A1 to inhibit proliferation and induce death of brain cancer cells and other cancer cells is based on the fact that protein phosphatase-2A1 is an enzyme which is involved in the control of cell maintenance, cell proliferation and cell death. Drugs that target the enzymes of the protein phosphorylation/dephosphorylation apparatus of the cell are mostly inhibitor compounds. The present invention relates to the use of a peptide compound (SYRAPRO-2000) which can act as an activator of protein phosphatase-2A1 enzyme. More particularly, the present invention relates to the preparation of a peptide compound (SYRAPRO-2000) and its use to activate protein phosphatase-2A1 enzyme in vitro and in intact cells. The present invention also relates to the use of a peptide compound (SYRAPRO-2000) to inhibit proliferation and induce death of brain cancer cells and other cancer cells. The present invention which is based on a published paper (Janon 2005) describes for the first time the use of an activating compound of a protein phosphatase to modulate the activity of a cell. In this invention, the peptide compound activator of protein phosphatase-2A1 (SYRAPRO-2000) is used to cause inhibition of cell proliferation and induction cell death of transformed T cells and brain cancer cells.

### BRIEF DESCRIPTION OF THE DRAWINGS

FIG. 1. shows the structure of the peptide compound (SYRAPRO-2000) that was synthesized and that activates protein phosphatase-2A1. Protein phosphatase-2A1 purified from CD⁴⁺ T cells as described in materials and method was assayed in the presence of various concentrations of SYRAPRO-2000. 100 percent protein phosphatase-2A1 activity is equivalent to 0.005 unit. Similar result were obtained in 3 independent experiments.

FIG. 2. shows the activation of protein phosphatase-2A1 by various concentrations of SYRAPRO-2000 and that SYRAPRO-2000 does not activate protein phosphatase-1, protein phosphatase-2B and protein phosphatase-2C. Protein phosphatase-1 (squares), protein phosphatase-2A1 (circles), protein phosphatase-2B (triangles) and protein phosphatase-2C (stars) were assayed in the presence of various concentrations of HIV-1 Vpr⁷¹⁻⁹⁶. 100 percent protein phosphatase activity is equivalent to 0.005 unit. Similar results were obtained in 3 independent experiments.

FIG. 3. shows that SYRAPRO-2000 causes the activation of protein phosphatase-2A1 in intact cells. Jurkat cells (a CD⁴⁺ T cell line) were grown in RPMI 1640 and 10% (v/v) fetal bovine serum. 1600 ml of cell suspension (10⁶ cells per ml) were split into two flasks and one was treated with SYRAPRO-2000 for 1 hour (circles) while the other was not (squares). The cells from each flask were collected by centrifugation, washed with 50 mM Imidazole-Cl pH 7.2 plus 150 mM NaCl and then homogenized in homogenization buffer as described in materials and methods. The homogenate from each flask was chromatographed on a DEAE Sepharose column and the eluted fractions were assayed for type 2A protein phosphatase activity as described in materials and method. Similar results were obtained in 3 independent experiments.

FIG. 4. shows that SYRAPRO-2000 causes death of Jurkat cells, a human transformed CD⁴⁺ T cell line. Panel A, Jurkat cells (a CD⁴⁺ T cell line) were grown in RPMI 1640 and 10% (v/v) fetal bovine serum. Cells (10⁶ cells per ml) were treated (+) or not (−) with 1000 nM SYRAPRO-2000. Cell viability was then determined by counting the cells following tryphan blue staining after 1, 2 and 4 hours of incubation. Panel B, Following treatment with SYRAPRO-2000 for 1, 2 and 4 hours as indicated, Jurkat cells (10⁶/ml) were washed with RPMI plus 2% fetal bovine serum and then deposited onto glass slides by cytospinning. Phase contrast images were then obtained with white light emission on a Zeiss Axioplan 2 imaging universal microscope (Thornwood, N.Y.). The images were captured and stored digitally with the aid of Northern Eclipse Version 6 software (Zeiss, Thornwood, N.Y.). Similar results were obtained in 3 independent experiments.

FIG. 5. shows that SYRAPRO-2000 causes death of HTB3 cells, a human glioblastoma cell line. Panel A, HTB-3 cells (a human glioblastoma cell line) were grown in DMEM and 5% (v/v) fetal bovine serum. Cells (10⁶ cells per ml) were treated (+) or not (−) with 2000 nM SYRAPRO-2000. Cell viability was then determined by counting the cells following tryphan blue staining after 4, 8 and 12 days of incubation. Panel B, Following treatment with or without SYRAPRO-2000 for 8 days, HTB-3 cells grown on slides were analyzed by phase contrast microscopy (Row A), by fluorescence microscopy following staining with FITC labeled annexin V (apoptotic cell death) (Row B) or propidium iodide (oncolytic cell death) on a Cells were analyzed on a Zeiss Axioplan 2 imaging universal microscope (Thornwood, N.Y.). Phase contrast images and Green (FITC labeled annexin V) and red (propidium iodide) fluorescence images were obtained. The images were captured and stored digitally with the aid of Northern Eclipse Version 6 software (Zeiss, Thornwood, N.Y.). The excitation wavelengths were 495 nm (FITC labeled-annexin V) and 536 nm (propidium iodide) and the emmission wavelengths were 520 nm (FDA), 520 nm (FITC labeled-annexin V) and 617 nm (propidium iodide). The images were filtered with 510-550 nm (FDA), 510-550 nm (FITC-labeled annexin V), and 590-610 nm (propidium iodide) band pass filters.

FIG. 6. shows that SYRAPRO-2000 causes death of SK—N—SH cells, a human neuroblastoma cell line. Panel A, SK—N—SH cells (a human neuroblastoma cell line) were grown in DMEM and 5% (v/v) fetal bovine serum. Cells (10⁶ cells per ml) were treated (+) or not (−) with 2000 nM SYRAPRO-2000. Cell viability was then determined by counting the cells following tryphan blue staining after 4, 8 and 12 days of incubation. Panel B, Following treatment with or without SYRAPRO-2000 for 8 days, SK—N—SH cells grown on slides were analyzed by phase contrast microscopy (Row A), by fluorescence microscopy following staining with FITC labeled annexin V (apoptotic cell death) (Row B) or propidium iodide (oncolytic cell death) on a on a Zeiss Axioplan 2 imaging universal microscope (Thornwood, N.Y.). Phase contrast images and Green (FITC labeled annexin V) and red (propidium iodide) fluorescence images were obtained. The images were captured and stored digitally with the aid of Northern Eclipse Version 6 software (Zeiss, Thornwood, N.Y.). The excitation wavelengths were 495 nm (FITC labeled-annexin V) and 536 nm (propidium iodide) and the emmission wavelengths were 520 nm (FDA), 520 nm (FITC labeled-annexin V) and 617 nm (propidium iodide). The images were filtered with 510-550 nm (FDA), 510-550 nm (FITC-labeled annexin V), and 590-610 nm (propidium iodide) band pass filters.

### DETAILED DESCRIPTION

### DESCRIPTION OF THE INVENTION

This invention relates to the preparation of a peptide compound (SYRAPRO-2000), its use as an activator of protein phosphatase-2A1 in vitro and in intact cells and its use for inhibiting cell proliferation and inducing death of cancer cells.

### EXAMPLE 1

The peptide compound (SYRAPRO-2000), was designed based on the assumption that it is derived from the C-terminus of HIV-1 Vpr which was suspected to be an activator of protein phosphatase-2A1. The structure of SYRAPRO-2000 is HFRIGCRHSRIGVTRQRRARNGASRS. SYRAPRO-2000 can be synthesized on an automated Solid Phase Peptide Synthesizer and the synthesized compound can activate purified protein phosphatase-2A1 in vitro.

### EXAMPLE 2

The effect of synthesized SYRAPRO-2000 on purified protein phosphatase-2A1 can be determined in vitro. Protein phosphatase-2A1 can be assayed using ³²P-labeled phosphorylase a as substrate in the presence of various concentrations of SYRAPRO-2000. Protein phosphatase-2A1 is activated in the presence of SYRAPRO-2000.

### EXAMPLE 3

The effect of synthesized SYRAPRO-2000 on protein phosphatase-2A1 can be determined in intact cells. Jurkat cells, a human CD⁴⁺ T transformed cell line. Jurkat cells are grown in RPMI 1680 medium supplemented with 5% (v/v) fetal bovine serum and antibiotics and treated or not with 1 μM of SYRAPRO-2000 for 60 minutes. Following treatment with SYRAPRO-2000, cells are harvested by centrifugation, washed and homogenized. A cell extract is prepared by centrifugation and loaded onto a DEAE Sepharose column which is washed and eluted with a liner gradient of buffer plus 0 mM NaCl to buffer plus 400 mM NaCl. The eluted fractions are then assayed using ³²P-labeled phosphorylase a as substrate. Protein phosphatase-2A1 from cells treated with SYRAPRO-2000 is higher than from cells not treated with SYRAPRO-2000.

### EXAMPLE 4

The inhibition of cell proliferation and induction of death of Jurkat cells, a human transformed CD⁴⁺ T cell line by SYRAPRO-2000 can be demonstrated. Jurkat cells are grown in RPMI 1680 medium supplemented with 5% (v/v) fetal bovine serum and antibiotics and treated or not with 1 μM of SYRAPRO-2000 for 1 hour, 2 hours and 4 hours. Following treatment with SYRAPRO-2000, cell viability are then determined by counting the cells following tryphan blue staining. The number of viable cells are less when cells are treated with SYRAPRO-2000. Following treatment with SYRAPRO-2000, cells are deposited onto glass slides by cytospinning. Phase contrast images are then obtained. Death of Jurkat cells can be observed following treatment with SYRAPRO-2000.

### EXAMPLE 5

The inhibition of cell proliferation and induction of death of HTB-3 cells, a transformed human glioblastoma cell line by SYRAPRO-2000 can be demonstrated. HTB-3 cells are grown in DMEM supplemented with 5% (v/v) fetal bovine serum and antibiotics and treated or not with 2 μM SYRAPRO-2000 for 8 days. Following treatment with SYRAPRO-2000, cell viability are then determined by counting the cells following tryphan blue staining. The number of viable cells are less when cells are treated with SYRAPRO-2000. Following treatment with SYRAPRO-2000, cells grown on slides by were analyzed by phase contrast microscopy and fluorescence microscopy following staining with FITC labeled annexin V (an indicator of apoptotic cell death) or propidium iodide (an indicator of oncolytic or necrotic cell death). Death of HTB-3 cells can be observed following treatment with SYRAPRO-2000.

### EXAMPLE 6

The inhibition of cell proliferation and induction of death of SK—N—SH cells, a transformed human neuroblastoma cell line by SYRAPRO-2000 can be demonstrated. SK—N—SH cells are grown in DMEM supplemented with 5% (v/v) fetal bovine serum and antibiotics and treated or not with 2 μM SYRAPRO-2000 for 8 days. Following treatment with SYRAPRO-2000, cell viability are then determined by counting the cells following tryphan blue staining. The number of viable cells are less when cells are treated with SYRAPRO-2000. Following treatment with SYRAPRO-2000, cells grown on slides by were analyzed by phase contrast microscopy and fluorescence microscopy following staining with FITC labeled annexin V (an indicator of apoptotic cell death) or propidium iodide (an indicator of oncolytic or necrotic cell death). Death of SK—N—SH cells can be observed following treatment with SYRAPRO-2000.

Materials and Methods

Preparation of Proteins and Peptides.

³²P-labeled phosphorylase a was prepared by phosphorylation of phosphorylase b with phosphorylase kinase as described in (Cohen 1988). ³²P-labeled casein was prepared by phosphorylation of casein with protein kinase A as described in (Tung 1986). Protein phosphatase-1 was purified from rabbit skeletal muscle as described in (Cohen 1988). Protein phosphatase-1 inhibitor-2 was purified from rabbit skeletal muscle as described in (Yang 1981). Protein phosphatase-2A₀ and protein phosphatase-2A₁ from pig brain and from pig liver were purified as described in (Tung 1985). Protein phosphatase-2A₀ and protein phosphatase-2A₁ from pig adipose tissue were highly purified by successive chromatography of pig subcutaneous adipose tissue extracts on DEAE Sepharose, Poly-L-lysine Agarose, Sephacryl S-300 HR and thiophosphorylase-a-Sepharose-4B as described in (Tung 1985). Protein phosphatase-2B was purified from pig brain as described in (Tung 1986). Protein phosphatase 2C was highly purified from pig liver by successive chromatography of pig liver extracts on DEAE Sepharose, Casein Agarose and Sephacryl S-300 HR. Peptide activator compound of protein phosphatase-2A1 (SYRAPRO-2000) was prepared by chemical synthesis on an automated Solid Phase Peptide Synthesizer and purified as described in (Azzi 1992). The molecular mass of the peptide activator compound of protein phosphatase-2A1 was confirmed by MALDI ToF MS. The sequence SYRAPRO-2000 is HFRIGCRHSRIGVTRQRRARNGASRS.

Assay of Protein Phosphatases.

Protein phosphatase-1 was assayed as described in (Cohen 1988). Protein phosphatase-2B was assayed as described in (Tung 1986). Protein phosphatase-2C was assayed as described in (Tung 1986) except Ca²⁺ and calmodulin were omitted. The assay of protein phosphatases-2A1 consisted of 0.02 ml of enzyme solution in 50 mM Imidazole-Cl pH 7.2, 0.2 mM EGTA, 0.1% (v/v) 2-mercaptoethanol and 1 mg/ml bovine serum albumin (Assay Buffer), 0.01 ml of inhibitor-2 at 600 nM in Assay Buffer, 0.01 ml of protamine at 60 μg/ml in Assay Buffer or 0.01 ml of SYRAPRO-2000 at different concentrations in Assay Buffer or 0.01 ml of Assay Buffer alone, 0.02 ml of ³²P-labeled phosphorylase a at 3 mg/ml in Assay Buffer containing 15 mM caffeine. The assay components were preincubated for 10 minutes prior to initiating the reaction with ³²P-labeled phosphorylase a. One unit of protein phosphatase activity is that amount of enzyme which catalyzes the release of 1 nmol of phosphate from ³²P-labeled substrate per min at 30° C.

Culture of Jurkat CD⁴⁺ T Cells and Purification of Protein Phosphatase-2A₁.

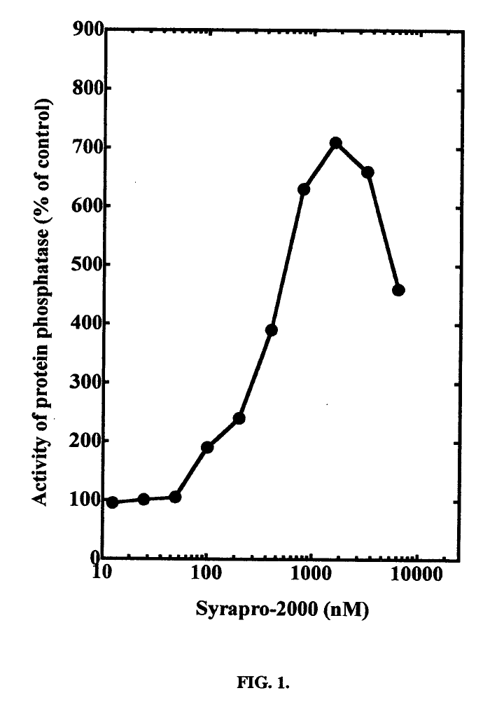

Jurkat cells, a human transformed CD⁴⁺ T cell line, were grown in grown in RPMI 1680 supplemented with 5% (v/v) fetal bovine serum at 37° C. in 95% air/5% CO₂ in a humidified incubator. 2.4 liters of cells were collected by centrifugation at 4200 rpm in a low speed centrifuge for 10 minutes. The cells were homogenized in 80 ml of 50 mM Imidazole-Cl pH 7.2, 2 mM EGTA, 2 mM EDTA, 0.1% (v/v) 2-mercaptoethanol, 0.2 mM PMSF, 1 mM benzamidine, 4 μg/ml aprotinin, 4 μg/ml leupeptin, 4 μg/ml pepstatin, 0.1 mM TLCK, 0.1 mM TPCK, 1 mM sodium orthovanadate and 10% (v/v) glycerol by 40 strokes in a glass hand held homogenizer. The homogenate was centrifuged at 29000 rpm in a high speed centrifuge for 30 minutes. The supernatant (i.e the extract) was collected and loaded onto a DEAE Sepharose column (1.5×6 cm) equilibrated in 25 mM Imidazole-Cl pH 7.2, 0.2 mM EGTA, 0.1% (v/v) 2-mercaptoethanol, 0.1 mM PMSF, 1 mM benzamidine and 10% (v/v) glycerol (Buffer A). The column was washed with 50 ml of Buffer and then eluted with a 200 ml linear gradient of Buffer A to Buffer A plus 0.4 M NaCl. The eluted fractions were then assayed for phosphorylase phosphatase activity in the presence of 100 nM inhibitor-2 and 10 μg/ml of protamine. Two major peaks of phosphorylase phosphatase were observed. The first and second peak represent protein phosphatase-2A₀ and protein phosphatase-2A₁ respectively. The second peak eluting at around 0.2 M NaCl, representing the largest proportion of the total protein phosphatase activity and which became activated following treatment of CD⁴⁺ T cells with HIV-1 Vpr⁷¹⁻⁹⁶ (FIG. 1), was collected and loaded onto a Sephacryl S-300 HR column (2.5×90 cm) equilibrated in 50 mM Imidazole-Cl pH 7.2, 0.2 mM EGTA, 0.1% (v/v) 2-mercaptoethanol, 0.2 M NaCl, 0.1 mM PMSF, 1 mM benzamidine and 10% (v/v) glycerol. The major activity eluting as a species of apparent molecular mass 300 kDa was collected, diluted four fold in Buffer A and loaded onto a poly-L-lysine Agarose Column (1.5×4 cm) equilibrated in Buffer A. The column was washed with 50 ml of Buffer A and then eluted with a 200 ml linear gradient of Buffer A to Buffer A plus 0.5 M NaCl. The active fractions eluting at ˜0.30 M NaCl salt concentration was pooled, concentrated by vacuum dialysis and stored at ⁻20° C. in 50 mM Imidazole-Cl pH 7.2, 0.2 mM EGTA in the presence of 50% (v/v) glycerol. The highly purified enzyme consisted of the characteristic subunits of protein phosphatase-2A₁, namely the A, B and C subunits. The purification of the enzyme is summarized in Table 1. The highly purified enzyme had a specific activity of 223.5 units per mg of protein. It is difficult to determine the activity of protein phosphatase-2A₁ in CD⁴⁺ T cell extract. However, assuming that it represented about 66 percent of total type 2A protein phosphatase activity in CD⁴⁺ T cell extract, the specific activity of the enzyme in extract was estimated to be 0.3 unit/mg. The enzyme was therefore purified 908 fold. Like other previously characterized forms of protein phosphatase-2A, protein phosphatase-2A₁ from CD⁴⁺T cells was not inhibited by inhibitor-2 but inhibited by okadaic acid and activated by protamine.

Culture of HTB-3 and SK—N—SH Cells.

HTB-3, a human glioblastoma cell line and SK—N—SH cells, a human neuroblastoma cell line were grown in DMEM supplemented with 5% (v/v) fetal bovine serum and antibiotics at 37° C. in 95% air/5% CO₂ in a humidified incubator.

Examination of Cell Viability and Cell Death in Cells Treated with SYRAPRO-2000.

Following treatment of cells with SYRAPRO-2000, cells were examined by phase contrast microscopy or by fluorescent microscopy following staining with FITC labeled annexin V (apoptotic cell death) and propidium iodide (oncolytic cell death). Cells were analyzed on a Zeiss Axioplan 2 imaging universal microscope (Thornwood, N.Y.). Phase contrast images were obtained with white light emission. Green (FITC labeled annexin V) and red (propidium iodide) fluorescence images were obtained. The images were captured and stored digitally with the aid of Northern Eclipse Version 6 software (Zeiss, Thornwood, N.Y.). The excitation wavelengths were 495 nm (FITC labeled-annexin V) and 536 nm (propidium iodide) and the emmission wavelengths 520 nm (FITC labeled-annexin V) and 617 nm (propidium iodide). The images were filtered 510-550 nm (FITC-labeled annexin V), and 590-610 nm (propidium iodide) band pass filters.

Results

Preparation of Peptide Compound Activator of Protein Phosphatase-2A1 (SYRAPRO-2000).

SYRAPRO-2000, a peptide compound activator of protein phosphatase-2A1 was prepared by chemical synthesis on an automated Solid Phase Peptide Synthesizer. Following synthesis, it was cleaved and purified by reverse phase HPLC on a C₁₈ column. The structure of SYRAPRO-2000 is: HFRIGCRHSRIGVTRQRRARNGASRS. The molecular mass of SYRAPRO-2000 was determined by MALDI ToF MS. The purified material, a white solid was readily dissolved in and reconstituted in water at ˜1-2 mg/ml. For its use as an in vitro activator of protein phosphatase-2A1, it is dissolved in the assay buffer which consisted of 0.02 ml of enzyme solution in 50 mM Imidazole-Cl pH 7.2, 0.2 mM EGTA, 0.1% (v/v) 2-mercaptoethanol and 1 mg/ml bovine serum albumin. For its use on intact cells, it is dissolved in the culture medium.

Effect of SYRAPRO-2000 on Purified Protein Phosphatase-2A1 in vitro.

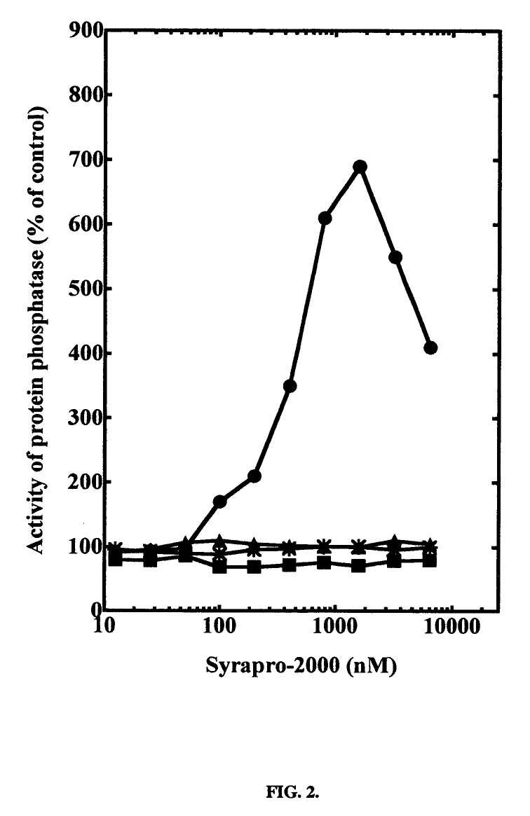

The effect of SYRAPRO-2000 on the activity of protein phosphatase-2A₁, the major form of protein phosphatase-2A in Jurkat cells, was determined in vitro. SYRAPRO-2000 activated protein phosphatase-2A₁ by 7 fold with half maximal activation occuring at around 300 nM. The effect of SYRAPRO-2000 was biphasic. At a concentration of above 1000 nM, there was inhibition of protein phosphatase-2A₁ by SYRAPRO-2000 (FIG. 1). The effect of SYRAPRO-2000 on the activities of other major forms of protein phosphatase namely, protein phosphatase-1, protein phosphatase-2B and protein phosphatase-2C were determined. FIG. 2 shows that the effect of SYRAPRO-2000 was quite specific. It had negligible effect on the activities of protein phosphatases-1, -2B and 2C.

Effect of SYRAPRO-2000 on Protein Phosphatase-2A1 in Intact Cells.

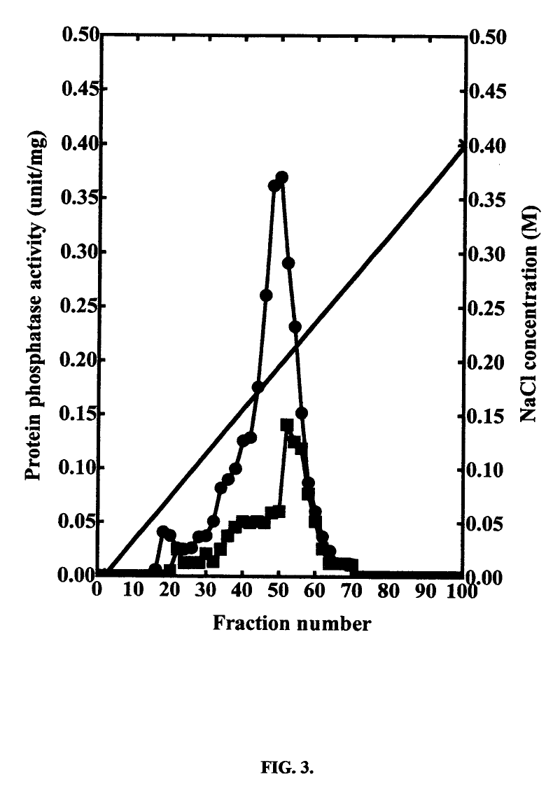

The effect of SYRAPRO-2000 on the activity of protein phosphatase-2A1 in intact cells was investigated. Jurkat cells, a CD⁴⁺ T cell line were grown in RPMI 1640 in 10% (v/v) fetal bovine serum and then treated or not with 1000 nM SYRAPRO-2000 which was added directly to the culture medium in the presence of 0.001% (v/v) DMSO. Extracts were then prepared as described in materials and methods section and chromatographed on DEAE Sepharose column, an efficient procedure for the separation of protein phosphatase-2A1. Protein phosphatases were then assayed in the presence of protein phosphatase-1 inhibitor-2, a very effective method for the determination of the different forms of protein phosphatase-2A (Tung 1985). As shown in FIG. 3, protein phosphatase-2A1 which elutes at around 0.18 to 0.2 M NaCl salt concentration became activated when cells were treated with SYRAPRO-2000.

Inhibition of Cell Proliferation and Induction of Cell Death in Jurkat Cells, a Human Transformed CD⁴⁺ T Cell Line.

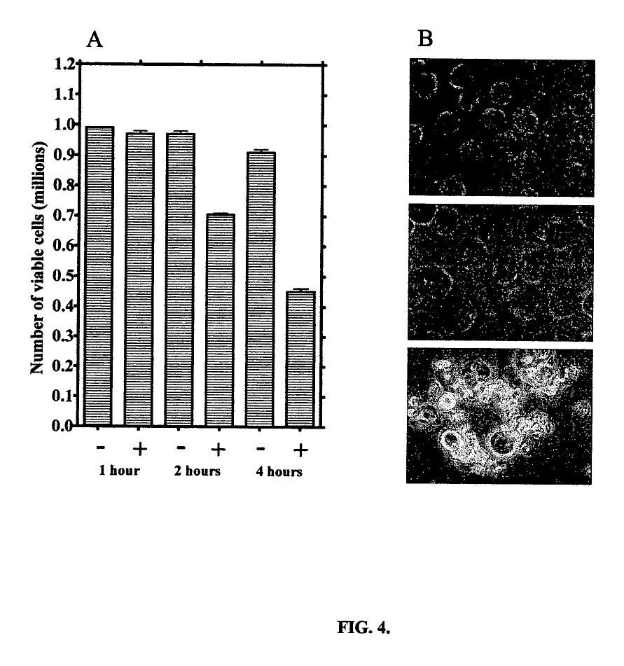

Treatment of Jurkat cells with SYRAPRO-2000 was accompanied by inhibition of cell proliferation and induction of cell death. After four hours of incubation with SYRAPRO-2000, almost 60 percent of the treated cells became non viable as determined by tryphan blue staining (FIG. 4A). Examination of CD⁴⁺ T cells treated with SYRAPRO-2000 by phase contrast microscopy showed that they died as a result of oncolysis (FIG. 4B).

Inhibition of Cell Proliferation and Induction of Cell Death in HTB-3 Cells, a Human Glioblastoma Cell Line.

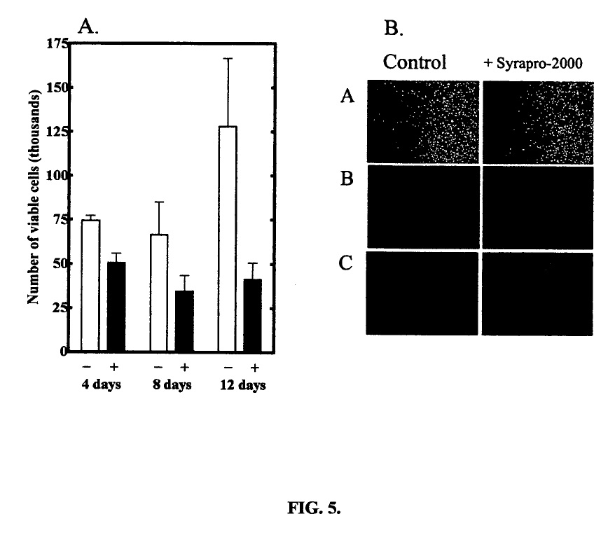

Treatment of HTB-3 cells with SYRAPRO-2000 was accompanied by inhibition of cell proliferation and induction of cell death. After 8 days of incubation with SYRAPRO-2000, almost 50 percent of the treated cells became non viable as determined by tryphan blue staining (FIG. 5A). Examination of HTB-3 cells treated with SYRAPRO-2000 by fluorescent microscopy following staining with FITC-annexin V and propidium iodide showed that they died as a result of apoptosis and oncolysis (FIG. 5B).

Inhibition of Cell Proliferation and Induction of Cell Death in SK—N—SH Cells, a Human Neuroblastoma Cell Line.

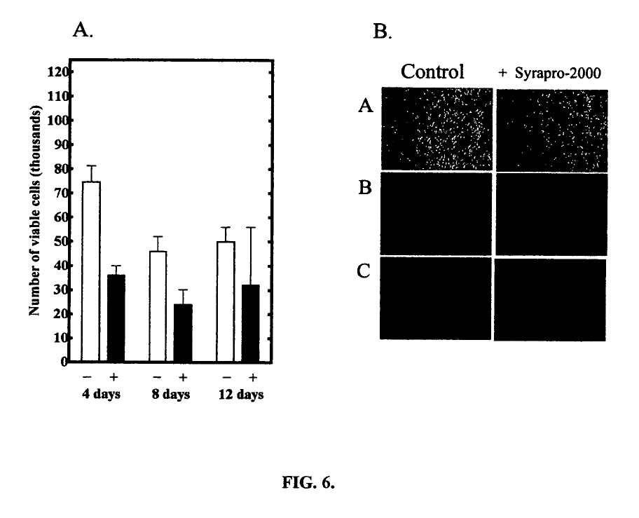

Treatment of SK—N—SH cells with SYRAPRO-2000 was accompanied by inhibition of cell proliferation and induction of cell death. After 4 days of incubation with SYRAPRO-2000, almost 50 percent of the treated cells became non viable as determined by tryphan blue staining (FIG. 6A). Examination of SK—N—SH cells treated with SYRAPRO-2000 by fluorescent microscopy following staining with FITC-annexin V and propidium iodide showed that they died as a result of apoptosis and oncolysis (FIG. 6B).

### REFERENCES CITED

Azzi, L., Meijer, L., Reed, S. I., Pidikiti, R. and Tung, H. Y. L. (1992). “Interaction between the cell-cycle-control poteins p34cdc2 and p9CKShs2. Evidence for two cooperative binding domains in p9CKShs2.” Eur. J. Biochem. 203: 353-360.

Chiang, C.-W., Kanies, C., Kim, K. W., Fang, W. B., Parkhurst, C., Xie, M., Henry, T. and Yang, E. (2003). “Protein phosphatase-2A dephosphorylation of phosphoserine 112 plays the gatekeeper role for BAD-mediated apoptosis.” Mol. Cell Biol. 23: 6350-6363.

Chiang, C. W., Harris, G., Ellig, C., Masters, S. C., Subramanian, R., Shenolikar, S., Wadszinski, B. E. and Yang, E. (2002). “Protein phosphatase-2A activates proapoptotic function of BAD in interleukin-3-dependent lymphoid cells by a mechansim requiring 14-3-3 dissociation.” Blood 2001(97): 1289-1297.

Cohen, P. (1989). “The structure and regulation of protein phosphatases.” Annu. Rev. Biochem. 58: 453-508.

Cohen, P., Alemany, S., Hemmings, B. A., Resink, T. J., Stralfors, P. and Tung, H. Y. L. (1988). “Protein phosphatase-1 and protein phosphatase-2A from rabbit skeletal muscle.” Methods Enzymol. 159: 390-409.

Cohen, P. (2002). “Protein kinase-the major drug targets of teh twenty-first century?” Nat. Rev. Drug Discov. 1: 309-315.

Deng, X., Ito, T., Carr, B., Mumby, M. and May, W. S. (1998). “Reversible phosphorylation of Bcl2 following interleukin-3 and bryostatin 1 is mediated by direct interaction with protein phosphatase-2A.” J. Biol. Chem. 273: 34157-34163.

Emerman, M. (1996). “HIV-1, Vpr and the cell cycle.” Curr. Biol. 6: 1096-1103.

Garcia, A., Cayla, X., Guergnon, J., Dassauge, F., Hospital, V., Rebollo, M. P., Fleisher, A. and Rebollo, A. (2003). “Serine/threonine protein phosphatases PP1 and PP2A are key players in apoptosis.” Biochimie 85: 721-726.

Ingebritsen, T. S., Stewart, A. A. and Cohen, P. (1983). “The protein phosphatases involved in cellular regulation. 6. Measurement of type-1 and type-2 protein phosphatases in extracts of mammalian tissues; an assessment of their physiological roles.” Eur. J. Biochem. 132: 297-307.

Ingebritsen, T. S. and Cohen, P. (1983). “The protein phosphatases involved in cellular regulation. 1. Classification and substrate specificities.” Eur. J. Biochem. 132: 255-261.

Ingebritsen, T. S. and Cohen, P. (1983). “Protein phosphatases: properties and role in cellular regulation.” Science 221: 331-338.

Jannssens, V. and Goris, J. (2001). “protein phosphatase-2A: a highly regulated family of serine/threonine phosphatases implicated in cell growth and signaling.” Biochem. J. 353: 417-439.

Janoo, A., Morrow, P. W. and Tung, H. Y. L. (2005). “Activation of protein phosphatase-2A₁ by HIV-1 Vpr cell death causing peptide in intact CD⁴⁺ T cells and in vitro.” J. Cell. Biochem. 94: 816-825.

Li, X., Yost, J., Virshup, D. M. and Seeling, J. M. (2001). “Protein phosphatase-2A and its B56 regulatory subunit inhibit wnt signalling in Xenopus.” EMBO J. 20: 4122-4131.

McCluskey, A., Sim, A. T. R. and Sakoff, J. A. (2002). Serine-threonine protein phosphatase inhibitors: Development of potential therapeutic strategies.” J. Med. Chem. 45: 1151-1175.

Meijer, L., Pondaven, P., Tung, H. Y. L., Cohen, P. and Wallace, R. (1986). “Protein phosphorylation and oocyte maturation II. Inhibition of starfish oocyte maturation by intracellular microinjection of protein phosphatases 1 and 2A and alkaline phosphatase.” Exp. Cell Res. 163: 489-499.

Melnikova, I. and Golden, J. (2004) “Targeting protein kinases.” Nat. Rev. Drug Discov. 3: 993-994.

Millward, T. A., Zolnierowicz, S. and Hemmings, B. A. (1999). “Regulation of protein kinase cascades by protein phosphatase-2A.” Trends Biochem. Sci. 24: 186-191.

Morrow, P. W., Tung, H. Y. L. and Hemmings Jr., H. C. (2004). “Rapamycin causes activation of protein phosphatase-2A1 and nuclear translocation of PCNA in CD⁴⁺ T cells.” Biochem Biophys. Res. Commun. 323: 645-651.

Pelech, S. and Cohen, P. (1985). “The protein phosphatases involved in cellular regulation. 1. Modulation of protein phosphatases-1 and 2A by histone H1, protamine, polylysine and heparin.” Eur. J. Biochem. 148: 245-251.

Petritsch, C., Beug, H., Balmain, A. and Oft, M. (2000). “TGF-b inhibits p70 S6 kinase via protein phosphatase-2A to induce G1 arrest.” Genes Dev. 14: 3093-3101.

Sontag, E., Federov, S., Kamibayashi, C., Robbins, D., Cobb, M., and Mumby, M. (1993). “The interaction of SV40 small tumor antigen with protein phosphatase-2A stimulates Map kinase pathway and induces cell proliferation.” Cell 75: 887-897.

Sontag, E., Sontag, J. M. and Garcia, A. (1997) “Protein phosphatase-2A is a critical regulator of protein kinase C signaling targeted by SV40 small t to promote cell growth and NF-kB activation.” EMBO J. 16: 5662-5671.

Tung, H. Y. L. (1986). “Phosphorylation of the calmodulin-dependent protein phosphatase by protein kinase C.” Biochem. Biophys. Res. Commun. 138: 783-788.

Tung, H. Y. L., Alemany, S. and Cohen, P. (1985). “The protein phosphatases involved in cellular regulation. 2. Purification, subunit structure and properties of protein phosphatase-2A₀, 2A₁ and 2A₂ from rabbit skeletal muscle.” Eur. J. Biochem. 148: 253-263.

Tung, H. Y. L., De Rocquigny, H., Zhao, L.-J., Cayla, X., Roques, B. P. and Ozon, R. (1997). “Direct activation of protein phosphatase-2A₀ by HIV-1 encoded protein complex NCp7:Vpr.” FEBS Lett. 401: 197-201.

Tung, H. Y. L., Resink, T. J., Hemmings, B. A., Shenolikar, S. and Cohen, P. (1984). “The catalytic subunits of protein phosphatase-1 and -2A are distinct gene products.” Eur. J. Biochem. 138: 635-641.

Yang, S. D., Vandenheede, J. R. and Merlevede, W. (1981). “A simplified procedure for the purification of the protein phosphatase modulator (inhibitor-2) from rabbit skeletal msucle.” FEBS Lett. 132: 293-295.

The inventor claims:

1. A method for the preparation of a peptide compound (SYRAPRO-2000) that is an activator of protein phosphatase-2A1.

2. Use of a peptide compound (SYRAPRO-2000) to activate protein phosphatase-2A1 in vitro.

3. Use of a peptide compound (SYRAPRO-2000) to activate protein phosphatase-2A1 in intact cells.

4. A method to activate protein phosphatase-2A1.

5. Use of SYRAPRO-2000 to activate protein phioosphatase-2A1, thereby inhibiting cell proliferation and causing death of brain cancer cells and other cancer cells.

6. Usage of activator compounds to activate protein phosphatase enzymes.
